(19) United States
(12) Patent Application Publication
(10) Pub. No.: US 2004/0241154 A1
(43) Pub. Date: Dec. 2, 2004

(54) Secretin for the treatment of asthma

(76) Inventors: Richard Jon Davis, Hertfordshire (GB); Kenneth Clark, Hertfordshire (GB)

Correspondence Address: NIXON & VANDERHYE, PC, 1100 N GLEBE ROAD, 8TH FLOOR, ARLINGTON, VA, 22201-4714, US

(21) Appl. No.: 10/484,922

(22) PCT Filed: Jul. 26, 2002
(86) PCT No.: PCT/GB02/03433

(30) Foreign Application Priority Data
Jul. 27, 2001 (GB) 0118383.9

(51) Int. Cl. A61K 38/54

(52) U.S. Cl. 424/94.2

(57) ABSTRACT

The invention is based on the finding that the secretin receptor is expressed in tissues present in the distal lung of humans. In patient with asthma, levels of the receptor are elevated compared to normal tissue. Treatment of tissue by secretin stimulates the movement of negative ions in the tissue. Addition of secretin to human bronchial tissue in vitro stimulates bronchorelaxation. The invention provides methods of treatment of asthma in a patient by administering to said patient an effective amount of an agent which triggers anion efflux and bronchorelaxation in respiratory tissue via the activation of a secretin receptor.

### FIELD OF THE INVENTION

The present invention relates to the treatment of asthma with or by activation of the hormone secretin or other secretin receptor ligands.

### BACKGROUND TO THE INVENTION

Asthma.

Asthma is an increasingly common condition which results from the interaction of genetic and environmental factors. Approximately 10% of children have asthma, usually as part of a syndrome of atopy, which is characterised by the presence of allergy, asthma, seasonal rhinitis and eczema. The incidence is lower in adults (˜5%), with significantly less demonstrating an atopic background. However the prevalence of this disease is steadily increasing, in part due to increased monitoring and detection.

Asthma is a chronic lung condition which has the hallmarks of reversible airflow obstruction, non-specific bronchial hyperreactivity and chronic airway inflammation (American Thoracic society, 1987). In the conducting airways there is increased thickness of the airway basement membrane due to smooth muscle hypertrophy/hyperplasia and a large inflammatory cell infiltrate. Extensive mucous and cellular debris from inflammatory cells and the airway epithelium can cause plugging of the bronchial airways which at its most severe can be fatal. The lung parenchyma however is relatively spared of these morphological and pathological changes.

A consequence of this airway remodelling in asthma may include incomplete reversible airway narrowing, bronchial hyper-responsivenesss, airway edema, and mucus hypersecretion. The direct cause of airway narrowing and hyperresponsiveness is unknown although it is generally proposed that abnormalities in the airway smooth muscle function results in decreased or impaired relaxation or increased contractility. Physiologically this leads to the characteristic symptoms of wheeze, cough and dyspnoea.

Current successful therapies utilised in the treatment of asthma, such as salbutamol, salmeterol, mediate bronchodilation of the airways via the stimulation of specific receptors which are coupled to the specific G-protein Gₛ, which in turn leads to an increase in the intracellular levels of the second messenger cAMP (Ng et al, 1999).

Secretin.

Secretin is a peptide hormone which is secreted from S cells in the proximal small intestine (especially the duodenum and jejunum) in response to acidic contents leaving the stomach. The structure of porcine secretin has been known for some time and it has been isolated from porcine intestine and has been found to be constituted by a peptide composed of 27 amino acid residues (Mutt et al, 1970). Moreover, it has been found that bovine and porcine secretins are identical, and are also similar to canine secretin.

Although bovine and porcine secretins behave identically with human secretin in some respects they are not structurally identical. These animal secretins differ from the human secretin at positions 15 and 16.

Secretin's physiological role is to stimulate water (H₂O) and bicarbonate (HCO₃⁻) secretion from the pancreas, leading to the neutralisation of acidic chyme. Its actions are mediated via a seven transmembrane domain, G protein coupled receptor (GPCR), a member of the glucagon-secretin-vasoactive intestinal peptide structurally related superfamily of GPCRs (IUPHAR Receptor Compendium, 1998), for which the peptide exhibits nanomolar affinity. Secretin receptor stimulation mediates increases in intracellular cAMP, and the activation of protein kinase A (PKA).

Secretin is currently approved by the FDA to diagnose gastrinoma and assess pancreatic function. Anecdotal reports from “off-label” use of secretin in paediatric autism suggest that it may improve both physiological and behavioural symptoms associated with autism, a disorder characterized by severely impaired communication, social skills and development (see for example WO98/52593, U.S. Pat. No. 6,020,310 or U.S. Pat. No. 6,020,314). In March 2000 Repligen Corporation (USA) announced it had initiated a Phase II clinical trial with secretin in children with autism, with the Phase II trial sites including the Mayo Clinic, the University of Rochester Medical Center and the Southwest Autism Research Center in collaboration with Phoenix Children's Hospital. Initial results of these trials suggest that secretin infusion may be beneficial in discrete groups of severely autistic children.

Secretin has also been proposed for the prophylaxis of the aspiration pneumonia syndrome (e.g. in EP0150760; AU3806485).

There are a wide number of reported synthetic and/or naturally occurring secretin peptide analogues and fragments (referred to herein as “secretin receptor ligands”) which exhibit a wide range of potencies, efficacies and selectivity for the secretin receptor. These include, but are not limited to mono/poly substituted secretin analogues, secretin fragments, substituted secretin fragments, reduced peptide bond analogues (Gardner et al, 1976; Gardner et al, 1979; Waelbroeck et al, 1981; Konig et al, 1984; Staun-Olsen et al, 1986; Robbertecht et al, 1988; Haffer et al, 1991), and naturally occurring and synthetic analogues, fragment and chimeric peptides of the VIP/secretin family (including VIP (vasoactive intestinal peptide), gastric inhibitory peptide (GIP), PACAP (pituitary adenylate cyclase-activating polypeptide), adrenomedullin, calcitonin, CGRP (alpha, beta and skin calcitonin gene related peptides), glucagon, glucagon-like peptide (GLP), growth hormone-releasing factor, parathyroid hormone (PTH) and its related protein (PTHrP), corticotrophin-releasing hormone (CRH) and amylin Many of these peptides (including glucagon, GLP, PACAP and VIP share significant amino acid homology, particularly in the amino terminus with secretin. All these peptides are though to adopt similar secondary structural characteristics, including one or two regions of amphipathic α-helical secondary structure, and appear to interact with their receptors in a well conserved manner (Sexton, 1999).

Also known are secretin-related receptor peptides, and associated analogues and fragments which exhibit affinity for the secretin receptor.

### DISCLOSURE OF THE INVENTION

We have studied the expression levels of secretin receptor in tissue from patients with asthma as well as healthy individuals. Secretin receptor mRNA has been demonstrated to be highly expressed within the human tertiary bronchus and lung parenchyma. These data were determined by quantitative real time PCR on ethically obtained human lung tissue. Function of this receptor within the lung has not been reported within the literature. Secretin receptor mRNA expression was determined in the human tertiary bronchus and parenchyma derived from 5 control and 5 asthmatic donors (see FIG. 2), and was found to be up-regulated in asthmatic tertiary bronchus.

The applicants have further found that addition of the human peptide secretin to the airway surface of the human tertiary bronchus in vitro, or to the luminal (airway) membrane of epithelial cells isolated and cultured from the human tertiary bronchus, stimulates ion movement consistent with that of a negatively charged ion, which is believed to be chloride.

To confirm this, the inventors have found that addition of secretin to epithelial cells isolated and cultured from the human tertiary bronchus is able to concentration dependently stimulate Cl⁻ ion efflux, with nanomolar potency (example 3).

These findings have been supported further by the additional demonstration that secretin stimulates relaxation of the precontracted human tertiary bronchial rings in vitro (example 4). This leads to the conclusion that secretin will exhibit a bronchorelaxant effect in vivo.

While not wishing to be bound by any one particular theory, the secretin receptor is coupled to the G-protein, Gₛ, and therefore it can be envisaged that activation of the functional secretin receptor that has been identified by the inventors on epithelial cells lining the distal human bronchus will result in the accumulation of intracellular cAMP, and subsequent bronchodilation. Consistent with this, recently Cl⁻ ion movement has been demonstrated to be linked to epithelium-dependent airway relaxation (Fortner et al, 2001), such that blockade of Cl⁻ ion secretion results in a significant reduction in agonist-induced relaxation. The inventors thus propose that secretin stimulated Cl⁻ ion efflux will further enhance bronchodilation mediated via secretin and other agonists (e.g. beta adrenergic agonists).

In addition, mucus hypersecretion and non-continuous clearance of tracheobronchial mucus also contribute to airflow obstruction in asthma, due to the formation of mucus plugs, which can be present simultaneously with airway responsiveness. Mucus plugging can result in small airway (e.g. tertiary bronchus) obstruction producing reduced maximal respiratory flow and slow forced lung emptying. In other mucus hypersecretory lung diseases, such as cystic fibrosis, reduction of predominantly Cl⁻ efflux alters the aqueous and ionic composition and subsequent viscosity of mucus and mucus secretions, leading to thick insipid mucus which impairs mucociliary clearance from the lung.

The inventors propose that endogenous or exogenous stimulation of the secretin receptor (e.g. via the addition of secretin, its mimetics or closely related peptides and analogues, or via a mechanism to enhance the release of endogenous secretin) will mediate relaxation of the airways via increasing intracellular concentrations of cAMP within epithelial cells lining the lower conducting airways; enhance agonist mediated relaxation of the airways, via the stimulation of Cl⁻ efflux; and/or enhance the clearance of mucus and mucus plugs of the airway by reducing the constitution of mucus via the stimulation of Cl⁻ efflux.

Accordingly, the present invention provides a method of treatment of asthma in a patient suffering from asthma, the method comprising administering to said patient an effective amount of an agent which triggers anion efflux and bronchial relaxation in respiratory tissue via the activation of a secretin receptor.

The present invention is in one part based on the surprising finding by the inventors of elevated levels of secretin receptor mRNA in the tertiary bronchus of asthmatic patients, and relates to the novel use of secretin in the treatment of asthma. A preferred aspect of the invention is directed to the treatment of asthma by the administration to the patient of a secretin receptor ligand. However, it has been contemplated by the inventors that secretin may be delivered to the patient in an effective amount by means other than directly administering the secretin receptor ligand itself. An alternative method of administering secretin is by the use of agents which stimulate the up-regulation of the production and or release of endogenous secretin in pulmonary cells, or secretin related peptides.

The invention also provides the use of an agent which triggers ion efflux in respiratory tissue via the activation of a secretin receptor for the manufacture of a medicament for the treatment of asthma.

Preferably, the agent is a secretin receptor ligand, more particularly secretin, particularly human secretin.

### BRIEF DESCRIPTION OF THE DRAWINGS

FIG. 1 shows an alignment of human, porcine and canine secretin.

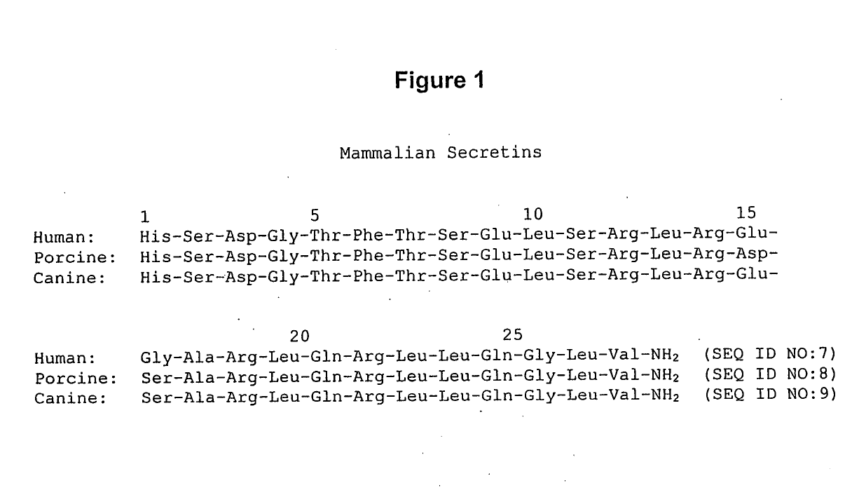

FIG. 2 shows secretin receptor mRNA expression appears increased in the asthmatic human tertiary bronchus compared with non-asthmatic controls. Data are presented as mean target copy threshold (Tcn) where the higher the Tcn, the lower the copy number of the gene.

FIG. 3 shows secretin mediates ionic movement in the human tertiary bronchus.

FIG. 4 shows secretin stimulates ionic movement in confluent monolayers of primary human bronchial epithelial cells (bold trace).

FIG. 5A shows that secretin 12.5 and 100 nM secretin stimulates Cl⁻ efflux from primary, human tertiary bronchial epithelial cells derived from non-asthmatic donors. FIG. 5B shows the results of a similar experiment using 1 (solid diamonds), 3 (solid circles, lower line), 10 (open squares), 30 (open triangles), 100 (open inverted triangles), 300 (open diamonds) nM and 1 μM (solid circles, upper line) secretin, and 1 μM PGE₂ (open squares, top).

FIG. 6 shows a concentration response curve of secretin stimulated Cl⁻ efflux from primary, human tertiary bronchial epithelial cells derived from non asthmatic donors (closed circles). The response to 1000 nM PGE2 is shown in the open circle.

FIG. 7 shows secretin stimulated relaxation of carbachol precontracted human bronchial rings. FIG. 7A shows a representative trace of the effects of secretin on carbachol pre-contracted human tertiary bronchus. FIG. 7B shows secretin mediates concentration-dependent relaxation of carbachol precontracted tertiary bronchial rings, in vitro, in terms of the absolute response in mN. FIG. 7C shows the same data as the % of isoprenaline (1 μM) relaxation.

### DETAILED DESCRIPTION OF THE INVENTION

Agent which triggers anion efflux in respiratory tissue via the activation of a secretin receptor.

There are a number of mechanisms by which secretin receptors may be activated. For example, expression of secretin is widely reported to be restricted to S-type enteroendocrine cells in the small intestine and colonic enteroendocrine cells and insulin producing β cells of the developing pancreas. Both enteroendocrine cells and pancreatic islets arise from the primitive embryonic gut endoderm. In addition, the primary airways are formed through a process termed branching morphogenesis, whereby two ventral lung buds sprout from the epithelium lining the floor of the embryonic foregut endoderm. Patterning of the airways is then accomplished by the outgrowth and repetitive branching of the two long buds. Pulmonary neuroendocrine (PNE) cells are amongst the first cells to differentiate from the primitive lung epithelium, and are generally most abundant in the airways of fetal and neonatal lungs. These cells are known to express a number of peptides including calcitonin, calcitonin gene related peptide, serotonin and endothelin, and can be visualized by their immunoreactivity to these peptides or to general endocrine markers such synaptophysin, chromogranin and protein gene product 9.5. In the CF bronchus, increased calcitonin immunoreactivity within endocrine cells has been demonstrated (Wolf et al, 1986).

Further, the secretin gene may be upregulated by the provision of agents which increase the level of transcription of the gene, e.g. via promoter or enhancer regulation. The enhancer region of the secretin gene contains a cis-acting DNA consensus sequence (CAGCTG) known as an E box, which bind proteins belonging to the basic helix-loop-helix (bHLH) family of transcription factors. A bHLH protein known as BETA2/NeuroD has been demonstrated to lead to the tissue-specific regulation of secretin gene transcription (Mutoh et al, 1997). In knock out mice, BETA2/NeuroD deficient mice fail to develop enteroendocrine cells or pancreatic β cells, demonstrating the critical role of this transcription factor in the normal development of several specialized cells types that arise from the gut endoderm. Beta2/NeuroD expression has been demonstrated to locate only to endocrine cells in transgenic mice (Rhindi et al, 1999).

In addition, up regulation of endogenous secretin production may also be achieved by a variety of other methods known in the art (e.g. see Jiang et al., 2001; Yang et al., 1998; Morse et al., 2001; Lewis et al., 1997; West & Rodman, 2001; Alton & Kitson, 2000) including but not limited to gene therapy (delivery of DNA or RNA in a viral or non viral vector encoding a peptide capable of directly or indirectly stimulating the secretin receptor or its cell signalling pathway), or gene targeting (delivery of agents which target regulatory sequences or transcription factor binding sites on the promoter region of the gene encoding secretin or a related peptide, thereby switching on production of secretin or a related peptide capable of directly or indirectly stimulating the secretin receptor).

A number of mechanisms are known to stimulate secretin release, including the following:

Agents such as dibutyryl cyclic-3′,5′-adenosine monophosphate, forskolin, 4 beta-12-O-tetradecanoylphorbol-13-acetate, the synthetic serine protease inhibitor, camostat, and the calcium ionophore, A2318, which stimulate Ca²⁺ and cyclic-3′,5′-adenosine monophosphate-dependent secretin release (Xue et al, 1993);

Pancreatic phospholipase A₂ (PLA₂) which has been demonstrated to intrinsically possess secretin-releasing activity, which is independent of its digestive enzymatic activity (Chang et al, 1999);

The neuropeptides bombesin, gastrin releasing peptide, VIP and galanin have also been shown to modulate secretin release in secretin-producing cells (Chang et al, 1998); and

Long chain fatty acids, such as sodium oleate are potent stimulators of secretin release from endocrine cells. Their stimulatory effect is potentiated by endogenous protein kinase A and mediated by activation of Ca²⁺ influx through the L-type channels and of protein kinase C and Ca²⁺/calmodulin-dependent protein kinase II (Chang et al, 2000).

Further, receptor activity modifying proteins, or RAMP are novel single transmembrane domain proteins that can modulate the expression and/or activity of at least two members of the secretin receptor GPCR family. To date there are 3 RAMP isoforms, 1-3, whose interactions are suggested to potentially result in trafficking of the receptor to the cell surface, modifying the degree of receptor glycosylation, and/or contributing to the ligand binding site through association with the receptor at the cell surface (Sexton, 1999).

RAMPS may indirectly alter a peptide selectivity for a specific receptor of the secretin GPCR family. For example, studies in which a single point mutation of the PTH1 receptor confers secretin responsiveness to this receptor, while the reverse mutation confers PTH responsiveness to the secretin receptor (Turner et al, 1996) has been suggested could be due to alterations in specific RAMP interactions with the receptor. (Sexton, 1999).

As such, agonism of the secretin receptor could be mediated via the simultaneous or sequential application of a peptide analogue or fragment of the secretin receptor family and a specific RAMP.

Respiratory tissue in which secretin receptors are activated particularly includes tissue within the distal regions of the lung selected from tertiary bronchus and lung parenchyma.

Secretin Receptor Ligand.

As indicated above, the preferred secretin receptor ligand is human secretin (hSN). However other mammalian secreting, such as the closely related bovine, porcine or pig secretin, or canine, rodent, chicken and rabbit secretin (which exhibit various degrees of homology to human secretin), may be used, as well other naturally occurring or synthetic fragments or analogues of secretin, such as those identified herein.

Various other secretin receptor ligands are well known in the art. Many such ligands are based on the sequence of a natural secretin (e.g. human or porcine secretin) but contain from 1 to 7 (more usually from 1 to 5, and often 1, 2 or 3) amino acid substitutions or deletions, particularly but not exclusively in the N-terminal region.

For example, Gespach et al (1986) describe four synthetic secretin analogues including one corresponding to porcine secretin substituted at the N-terminus by sequence portions of vasoactive intestinal peptide (VIP), i.e. Ala4-Val5-pSN, together with Tyr1-Ala2-Glu3-pSN, Gln3-pSN, Phe1-Phe2-Trp3-Lys4-pSN. Konig et al (1977) describe Ala4-pSN. Gardener et al (1976) describe the secretin fragment SN5-27 and three variants thereof, (9Gln-SN5-27, 15Asn-SN5-27 and 9Gln-15Asn-SN5-27). 15-Lys-SN has also been described in the art (Gardener et al, 1979). Haffer et al (1991) describe eight secretin variants with reduced peptide bonds (the —CONH— bond being replaced by —CH2—HN—) between one of the eight N-terminal peptide bonds. Robberecht et al (1988) describe secretin fragments 2-27, 3-27, 5-27 and 7-27 and observed activity for secreting receptors. Konig et al (1986) exchanged the N-terminal 5 amino acids of a secretin for the N-terminal pentapeptide sequence of human somatotropin releasing factor to provide 1-Tyr-2,4-diAla-5-Ile-SN, which showed secretin activity. Other active variants made were 3-L-Cystic acid-SN, 6-D-Phe-SN, 5-Allo-Thr-SN, and 1-Cys-6-Cys-SN.

Further examples of secretin analogues which exhibit affinity for the secretin receptor include, [Ala4, Val5] and [D-Ala4,Val5]secretin, (D-Ala4) secretin; (D-Phe6) secretin; secretin 5-27, secretin 14-27 [Val5]secretin, [D-Ala4, Val5]secretin (Waelbroeck et al, 1981); substituted fragments such as [Gln9,Asn15]secretin (5-27) (Staun-Olsen et al, 1986); phenolic group containing analogues of porcine secretin including Nalpha-tyrosylsecretin, [Tyr1]secretin, and Nalpha-beta-(4-hydroxyphenyl) propionylsecretin (Yanaihara et al, 1977); carboxyl-terminal tricosapeptide analogues of secretin (S5-27) (9-Gln-S5-27, 15-Asn-S5-27), and 9-Gln-15-Asn-S5-27) (Gardner et al, 1976).

Vasoactive intestinal peptide (VIP), PACAP, glucagon, glucagon-like peptide and naturally occurring and synthetic analogues and fragments thereof, exhibit considerable homology to that of secretin. Examples of these include but are not limited, to (D-Ala4) VIP; (D-Phe4) VIP; (D-Phe2)VIP, fatty acyl derivatives of VIP, including myristyl-, palmityl- and stearyl-[Nle17]VIP (Gourlet et al, 1998), VIP 2-28; VIP 1-14; VIP 2-14; VIP 14-28; VIP 15-28; VIP 20-28; VIP 21-28, two sequences where the N-terminal VIP 1-6 or VIP 1-9 have been joined covalently with the C-terminal VIP 20-28 or VIP 21-28 (Couvineau et al, 1984); VIP 7-27, VIP 11-28, VIP 1-22-NH₂, VIP 16-28 (Staun-Olsen et al, 1986), VIP[10-28] and VIP[16-28]. Analogues of secretin and VIP, referred to as the vasectrins, have also been described by Beyerman et al, 1981. PACAP (1-27; 1-38) and analogue examples include PACAP(1-23, VIP-24-28), PACAP(1-24,Cys-25), PACAP(1-23), PACAP(3-27), PACAP(1-19), PACAP(3-19), PACAP(1-12), and PACAP(18-38) (Schmidt et al, 1993), Glucagon, and GLP-1, and their related analogues and fragments include GLP-1 (7-37) GLP-1-(1-37) amide, -(6-37) amide, -(8-37) amide, -(7-36) amide (Suzuki et al, 1989), those with alterations in the N-terminal position 1 including N-methylated- (N-me-GLP-1), alpha-methylated (alpha-me-GLP-1), desamidated- (desamino-GLP-1) and imidazole-lactic-acid substituted GLP-1 (imi-GLP-1). (Gallwitz et al, 2000).

The secretin receptor ligands described in the above literature, which is incorporated herein by reference, may all be used in the present invention, though those of skill in the art will appreciate that the above-cited references are not exhaustive and other secretin receptor ligands may be used.

The suitability of candidate ligands may be determined experimentally. For example, Charlton et al (1983) report that secretin injected intracerebroventricularly significantly increased defecation and decreased novel-object approaches in rats, but showed no significant effects on stereotypic behaviour. Such a test may be performed in rats with a secretin receptor ligand to determine its suitability for the present invention (i.e. those ligands which show similar effects via agonism of the secretin receptor may be selected).

Secretin is available from commercial sources (e.g. Peninsula Laboratories Inc, USA) or it and the above-described ligands may be obtained by reference to readily available published literature.

Compositions of the Invention.

The novel findings reported herein give rise to novel compositions which comprise a secretin receptor ligand together with at least one other compound active against asthma, generally agents which produce airway relaxation or enhance mucus clearance.

Such compounds include but are not limited to beta-agonist (e.g. salbutamol, salmeterol), disodium cromoglycate, steroids and inhibitors of PDE_{IV}.

The amount of secretin receptor ligand in such a composition may be, for example, from 1% to 99% by weight of the total amount of active ingredients (i.e. excluding carriers or diluents), for example from 10% to 90% by weight.

In a related aspect, the present invention provides a combination of a secretin receptor ligand and a second compound active against asthma for simultaneous or sequential use in the treatment of asthma. By “simultaneous” it is meant that the two compounds are administered at the same time, though not necessarily in the same composition. By “sequential” it is meant that the two compounds are administered within a time period such that the first of the two compounds is still active in the patient when administration of the second of the two compounds occurs. Preferably, “sequential” means within the same 24 hour, preferably within the same 12 hour, such as within the same 6, 3, 1, half or quarter hour time period.

Formulation and Administration.

Treatment of patients in accordance with the present invention may be performed by administering to a patient a secretin receptor ligand in the form of a pharmaceutical composition, either with or without a further active ingredient present (reference below to compositions will be understood to include both types, though for brevity only the secretin receptor ligand is specifically mentioned). The composition may be in combination with a non-toxic, pharmaceutically acceptable carrier. In this context the invention also covers a method of treating asthma comprising administering a therapeutically effective amount of the secretin receptor ligand of this invention or a composition of this invention on a patient to be treated.

In clinical practice the compositions of the present invention may be administered parenterally due to the fact that being a peptide the hormone is sensitive to biologically active environments. Oral or rectal administration may, however, be conceivable, for example using compositions of the slow release type making it possible for the active ingredient to reach the site of primary interest, namely the tertiary bronchus.

Secretin receptor ligands may be formulated in a suitable form for administration by inhalation (e.g. via an aerosol) or insufflation (either through the mouth or nose), or by parenteral administration (introduced by routes other than intestinal routes).

Delivery of proteins or peptides via inhalation may be accomplished using liquid or solid preparations of the secretin receptor ligand. Thus the invention contemplates formulations comprising secretin receptor ligand for us in a wide variety of devices that are designed for the delivery of pharmaceutical compositions and therapeutic formulations to the respiratory tract. In one aspect of the present invention, secretin receptor ligand is administered in aerosolized or inhaled form. The secretin receptor ligand, combined with a dispersing agent, or dispersant, can be administered in an aerosol formulation as a dry powder or in a solution or suspension with a diluent.

Suitable dispersing agents are well known in the art, and include but are not limited to surfactants and the like. Surfactants are generally used in the art to reduce surface induced aggregation of protein caused by atomization of the solution forming the liquid aerosol. Examples of such surfactants include polyoxyethylene fatty acid esters and alcohols, and polyoxyethylene sorbitan fatty acid esters. Amounts of surfactants used will vary, being generally within the range of about 0.001 to 4% by weight of the formulation. In a specific aspect, the surfactant is polyoxyethylene sorbitan monooleate or sorbitan trioleate.

The liquid aerosol formulations contain the secretin receptor ligand and a dispersing agent in a physiologically acceptable diluent. The dry powder aerosol formulations of the present invention consist of a finely divided solid form of the secretin receptor ligand and a dispersing agent, and optionally a bulking agent, such as lactose, sorbitol, sucrose, or mannitol, and the like, to facilitate dispersal of the powder. With either the liquid or dry powder aerosol formulation, the formulation must be aerosolized. That is, it must be broken down into liquid or solid particles in order to ensure that the aerosolized dose actually reaches the bronchi and/or alveoli, as desired. In general the mass median dynamic diameter will be 5 micrometers (μm) or less in order to ensure that the drug particles reach the lung bronchi or alveoli (Wearley et al 1991).

With regard to construction of the delivery device, any form of aerosolization known in the art, including but not limited to nebulization, atomization or pump aerosolization of a liquid formulation, and aerosolization of a dry powder formulation, can be used in the practice of the invention. A delivery device that is uniquely designed for administration of solid formulations is envisioned. Often, the aerosolization of a liquid or a dry powder formulation will require a propellant. The propellant can be any propellant generally used in the art. Examples of useful propellants include chlorofluorocarbons, hydrofluorocarbons, hydrochlorofluorocarbons, and hydrocarbons, including trifluoromethane, dichlorodifluoromethane, dichlorotetrafluoroethanol, and 1,1,1,2-tetrafluoroethane, and combinations thereof.

In a preferred aspect of the invention, the device for aerosolization is a metered dose inhaler. A metered dose inhaler provides a specific dosage when administered, rather than a variable dose depending on administration. Such a metered dose inhaler can be used with either a liquid or a dry powder aerosol formulation.

Systems of aerosol delivery, such as the pressurized metered dose inhaler and the dry powder inhaler are disclosed in Newman, Aerosols and the Lung, Clarke, S. W. and Davia, D. editors, pp 197-22 and can be used in connection with the present invention.

Additional pharmaceutical methods may be employed to control the duration of action of the antagonists of this invention. The antagonists also may be entrapped in microcapsules prepared, for example, by coacervation techniques by interfacial polymerisation (for example, hydroxymethylcellulose or gelatin-microcapsules and poly-(methylmethacrylate)microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Osol, A., ed (1980).

For intranasal administration, the secretin receptor ligands may be formulated as solutions for administration via a suitable metered or unit device or alternatively as a powder mix with a suitable carrier for the administration using a suitable delivery device. Alternatively, secretin receptor ligands could be delivered transnasally in a similar fashion. For example, preparation of secretin for transnasal administration has been described in JP60123426.

Preparations for parenteral administration includes sterile aqueous or non-aqueous solutions, suspensions or emulsions. Examples of non-aqueous solvents or suspending media are propylene glycol, vegetable oils, such as olive oil, and injectible organic esters, such as ethyl oleate. These compositions may also contain adjuvants, such as preserving, wetting, emulsifying and dispersing agents. They may be sterilized, for example, by filtration through a bacteria-retaining filter, by incorporation of sterilizing agents in the composition, by irradiation or by heating. They may be also be manufactured in the form of sterile solid compositions, which can be dissolved in a sterile injectible medium immediately before use. As well as the more customary intravenous and intramuscular routes the compositions may also be administered by intraarticular injection.

The percentages of active ingredient in the compositions of the invention may be varied as long as they constitute a proportion such that a suitable dosage for the desired stimulatory effect on the pancreas is obtained. Obviously several unit dosage forms may be administered at about the same time. Generally, the compositions should contain from about 0.1% to about 80% by weight of active ingredient.

The dose employed depends upon the desired stimulatory effect, the route of administration and the duration of the treatment. Typical doses may be in the range of from 10⁻⁸ to 10⁻³ mg per day, preferably from 10⁻⁶ to 10⁻⁴ mg per day for a human patient. The secretin receptor ligand may be administered each day or, according to the wishes of the medical practitioner, less often, e.g. weekly, or until the desired therapeutic effect is achieved.

### EXAMPLES

### Example 1

### RNA Expression Profiles

Messenger RNA expression profiles of the secretin receptor (protein accession P47872; nucleotide accession U28281) was examined. Total RNA was isolated from tertiary/quaternary bronchus and lung parenchyma from 5 control and 5 asthma donors using TriZol™ a commercially available solution of phenol and guanidine isothiocyanate, according to the protocol described by the manufacturer (Life Technologies). Samples of RNA were used only if intact 18s and 28s ribosomal RNA were detected by gel electrophoresis and if genomic DNA formed less than 10% of the total nucleic acid sample. Total RNA samples were annealed to the primer probe sequence plus a glyceraldehyde-3-phosphate dehydrogenase (GAPDH; accession no. P04406) primer and reverse transcribed using MuLV reverse transcriptase. Quantitative sequence detection was carried out on the resulting cDNA.

The applicants have developed protocols for quantitative analysis of mRNA expression using the ABI prism 7700 Sequence Detection System (Perkin Elmer). Details of the system are set out in WO00/05409. In brief, the system uses fluorogenic probes to generate sequence specific fluorescent signals during PCR. The probes are oligonucleotides with fluorescent reporter and quencher dyes attached. While a probe is intact, the intensity of reporter fluorescence is suppressed by a quencher. When a probe forms part of a replication complex during the PCR process, the quencher is separated from the reporter dye resulting in a increase in fluorescence which is then detected by the ABI 7700 sequence detector. The ABI 7700 has a built in thermal cycler, and a laser directed at each of the 96 sample wells via bi-directional fibre optic cables. Emitted fluorescence through the cables to a detector where emissions which fall between 520 nm and 660 nm are collected every few seconds. The system software analyses the contribution of each component dye to the experiment spectrum, and normalises the signal to an internal reference dye. The peaks of these normalised ‘reporter’ values (Rn) are then plotted against thermal cycle number to produce an amplification plot—to allow visualisation of the extent of PCR product generation.

The starting copy number of a target sequence (Cn) is established by determining the-fractional PCR cycle number (Ct) at which a PCR product is first detected—the point at which the fluorescence signal exceeds a threshold baseline. Therefore the lower a Ct value the greater the Cn.

Quantification of the amount of target mRNA in each sample is established through comparison of the-experimental Ct values with standard curves for the target sequence which are constructed during each experiment.

Primer probe sets were specifically designed for the detection of secretin receptor mRNA. Off-line homology searches revealed no significant matches with gene sequences logged at Genbank. Forward and reverse primer and probe sequences for the secretin receptor were as follows:

| | | |
|---|---|---|
| Forward | GACCAGCATCATCTGAGAGGCT | (SEQ ID NO: 1) |
| Reverse | CCTTCGCAGGACCTCTCTTG | (SEQ ID NO: 2) |
| Probe | TCTCTGTCCGTGGGTGACCCTGCT | (SEQ ID NO: 3) |

GAPDH primer probe sets were as follows

| | | |
|---|---|---|
| Forward | GAAGGTGAAGGTCGGAGTCAAC | (SEQ ID NO: 4) |
| Reverse | CAGAGTTAAAAGCAGCCCTGGT | (SEQ ID NO: 5) |
| Probe | TTTGGTCGTATTGGGCGCCT | (SEQ ID NO: 6) |

Reaction conditions were optimised using genomic DNA as a template and a primer probe concentration grid followed by a probe concentration gradient experiment. Primer concentrations were selected to give the most efficient amplification of gene product, i.e. those which generate a low threshold cycle and a relatively high accumulation of fluorescence. These optimal primer concentrations were then used to select the optimum probe concentration

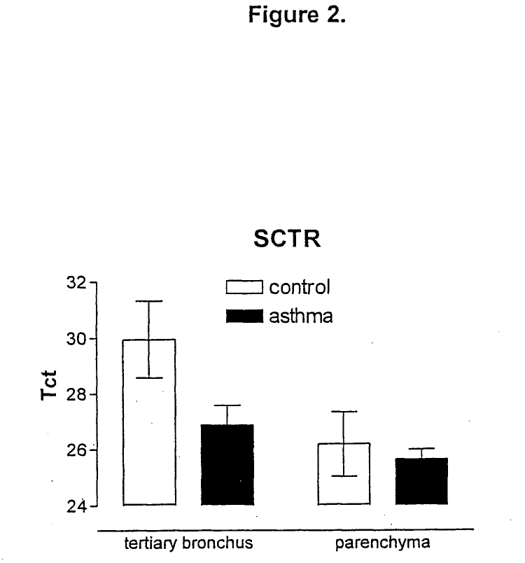

The data from 5 patients with asthma and 5 normal controls are shown in FIG. 2. Data are expressed as mean ± s.e.mean of the target threshold cycle (TCt), whereby a lower threshold cycle represents an increased mRNA abundance.

### Example 2

### Functional Activity of Secretin Receptor in Tertiary Bronchus

Functional activity of the secretin receptor was examined in the tertiary bronchus and in epithelial cells derived from the tertiary bronchus of normal tissue.

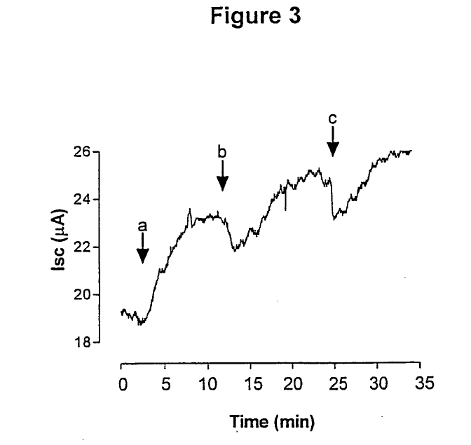

In brief, non-branching regions of the human tertiary bronchus from non-asthma donors were dissected, cut longitudinally and mounted in between the two compartments of a modified Ussing chamber to measure the short circuit current across the bronchial wall. Both luminal (airway) and basolateral membranes were bathed in oxygenated Krebs extracellular solution and the tissue voltage clamped to zero to allow changes in short circuit current in response to secretin to be measured. Amiloride at a concentration of 10 μM was initially added to the luminal membrane (FIG. 3, point a) (as described by those in the art) to partially block the predominant sodium ion current and unmask underlying ionic currents. On attainment of a stable base line, 3 μM human secretin (supplied by Sigma, catalogue number S714) was added to the luminal membrane (FIG. 3, point b).

Secretin was found to stimulate ionic movement in a manner consistent with the movement of a negatively charged ion (Cl⁻ and/or HCO₃⁻ (FIG. 3). Like secretin, addition of 10 μM ATP or UTP to the apical membrane of the lung epithelium (FIG. 3, point c) was demonstrated to stimulate a similar ionic movement of similar magnitude. These ATP and UTP mediated effects are widely reported in the literature to be due to the stimulation of a Ca²⁺-activated Cl⁻ current via the P2Y2 purinoceptor. Both described agonists, at high concentrations produced responses of a similar magnitude.

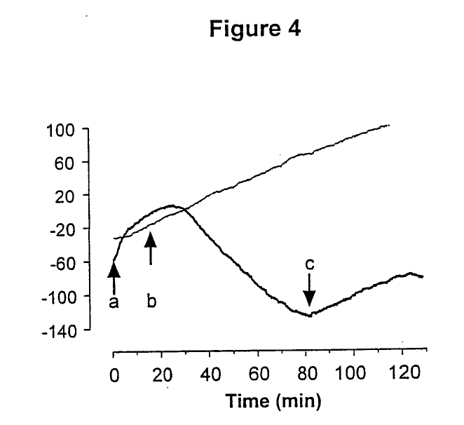

Functional effects of the secretin receptor were probed in epithelial cells derived from the human tertiary bronchus. In brief, tertiary bronchial epithelial were isolated by overnight protease digestion and then cultured until confluency on Snapwell (Costar) permeable supports. The supports were mounted in a modified Ussing chamber, and both luminal and basolateral membranes were bathed in oxygenated Krebs extracellular solution. The cells were voltage clamped to zero to allow changes in short circuit current in response to secretin to be measured. As previously described, 10 μM amiloride was initially added to the luminal membrane (FIG. 4, point a) followed by the addition of 100 nM secretin to the luminal membrane (FIG. 4, point b). Consistent with observations in the tertiary bronchus, secretin stimulated ionic movement in a manner consistent with the movement of a negatively charged ion (Cl⁻ and/or HCO₃⁻). Furthermore, addition of 500 μM glibenclamide (point c), a recognised inhibitor of the CFTR failed to suppress secretin mediated ionic movement, suggestive that a similar ionic movement would be observed in CF tertiary bronchial epithellal cells.

### Example 3

### Chloride Efflux From Human Tertiary Bronchial Cells

### Example 3a

### 12.5 and 100 nM Secretin

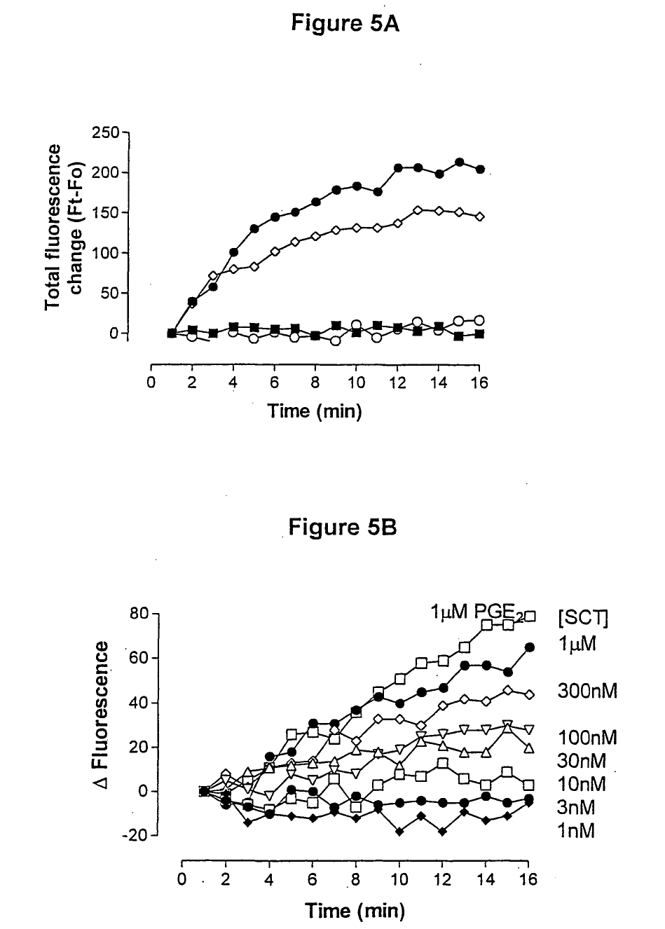

Ionic movement in tertiary bronchial epithelial cells was further characterised with the use of the Cl⁻ specific fluorescent probe MQAE (n-(ethoxycarbonylmethyl)-6-methoxyquinolinium bromide; Molecular Probes). In brief, primary human, tertiary bronchial epithelial cells were isolated as described above and cultured in a 96 well plate. On reaching confluency, cells were loaded overnight with 4 mM MQAE. Cells were washed in a chloride containing HEPES buffer, before passive Cl⁻ efflux was initiated by the addition of a Cl⁻ free buffer. Addition of 12.5 nM or 100 nM concentrations of secretin stimulated Cl⁻ efflux, as determined by changes in MQAE fluorescence. The results are shown in FIG. 5A (open diamonds 12.5 nM; closed circles 100 nM secretin). The 100 nM secretin mediated changes in fluorescence were abolished by the addition of 100 μM of the -non-selective Cl⁻ channel blocker NPPB (5-nitro-2-(3-phenylpropyl-amino)benzoic acid (open circles) . Unstimulated Cl⁻ efflux is demonstrated by the closed squares.

### Example 3b

### 1 nM to 1 μM Secretin and 1 μM PGE₂

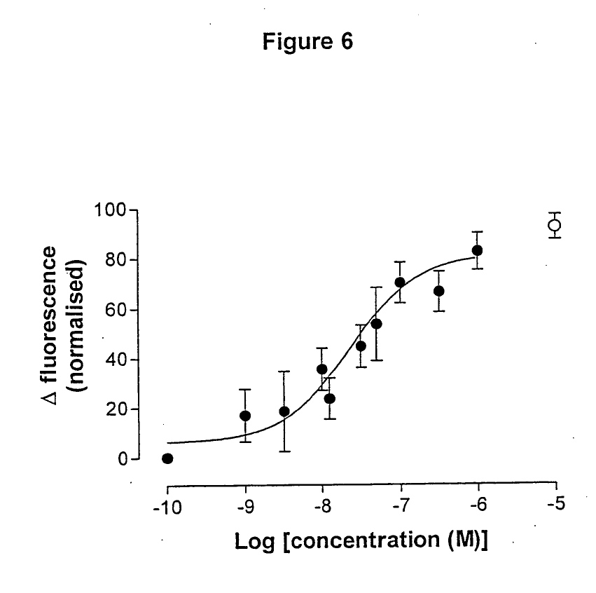

The experiment of example 3a above was repeated using a range of secretin concentrations from 1 to 1000 nM. Representative data from a single experiment from 6 independent experiments are shown in FIG. 5B. Maximal secretin stimulated Cl⁻ efflux was of a similar magnitude to that observed to Prostaglandin E₂ (10 μM). Secretin concentration-dependently stimulated Cl⁻ efflux with nanomolar affinity (Log EC₅₀ (M)-7.64±0.24; 22.9 nM—see FIG. 6). Responses at each concentration were normalised to those observed to PGE₂ (FIG. 5a open circles, 10 μM) and are representative of the mean ± s.e.mean from 2 to 6 independent experiments on cells derived from 6 donors).

### Example 4

### Bronchorelaxant Properties of Secretin

The bronchorelaxant properties of secretin was examined in vitro, in the tertiary bronchus of non-diseased tissue. In brief, tertiary bronchial rings (1-3 mm internal diameter) were mounted in 10 ml organ baths under a resting tension of 10 mN (1 g) and bathed with oxygenated Krebs' solution at 37° C. Tissues were precontracted with carbachol at the concentration being that which caused a contraction approximately 60-70% of the maximum, and a cumulative concentration-effect contractile curve (CEC) to secretin (0.01 μM to 3 μM) was constructed on each preparation. Isoprenaline (1 μM) was subsequently administered to demonstrate maximal relaxatory responses.

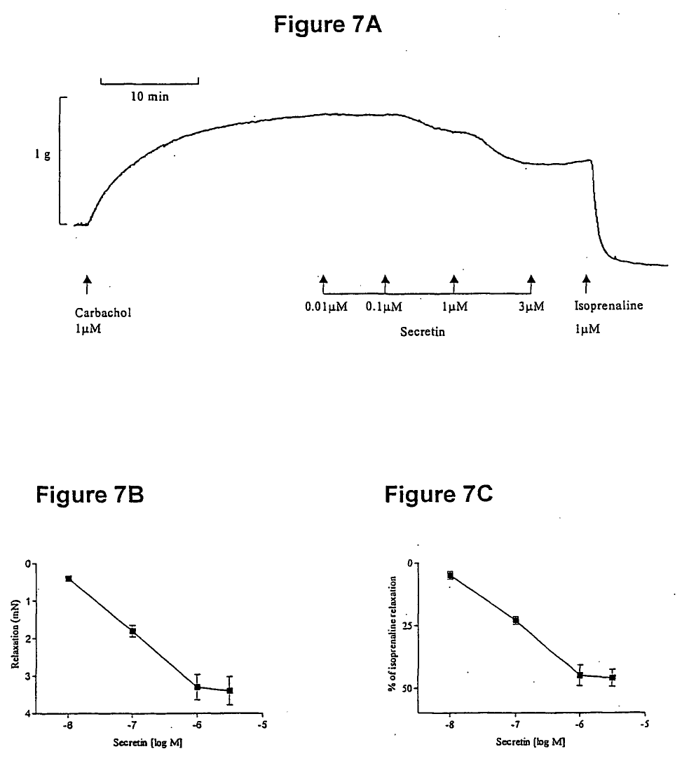

Secretin (0.01 μM to 3 μM) caused concentration-dependent relaxations of the carbachol-evoked tone (FIG. 7) in 7 of the 8 preparations tested, with a mean relaxation at 3 μM which was equivalent to 37±11% of the relaxation observed to isoprenaline (1 μM; n=7 preparations from 4 donors). The mean pEC₅₀ for secretin was 6.8±0.2.

### References

Beyerman, H. C., Buijen van Weelderen, A. W., Chang, T. M., Chey, W. Y., Grossman, M. I., Kranenburg, P., Scratcherd, T., Solomon, T. E., Voskamp, D. (1981) Synthesis, biological and immunochemical properties of analogues of secretin and vasoactive intestinal peptide (VIP): the vasectrins. Life Sci. 29:895-902

Chang, C. H., Chey, W. Y., Chang, T. M. (2000). Cellular mechanism of sodium oleate-stimulated secretion of cholecystokinin and secretin. Am. J. Physiol. Gastrointest. Liver. Physiol. 279:G295-303

Chang, C. H., Chey, W. Y., Erway, B., Coy, D H, Chang T M (1998). Modulation of secretin release by neuropeptides in secretin-producing cells. Am J Physiol 275; G192-202

Chang, T., Chang, C. H., Wagner, D. R. & Chey, W. (1999) Porcine Pancreatic Phospholipase A₂ Stimulates Secretin Release from Secretin-producing Cells. J. Biol. Chem. 274; 10758-10764

Charlton, C. G., et al (1983) Secretin modulation of behavioural and physiological functions in the rat. Peptides, 4; 73942.

Couvineau, A., Rouyer-Fessard, C., Fournier, A., St Pierre, S., Pipkorn, R., Laburthe, M. (1984) Structural requirements for VIP interaction with specific receptors in human and rat intestinal membranes: effect of nine partial sequences. Biochem Biophys Res Commun. 121:493-8

Fortner, C. N., Lorenz, J. N. & Paul, R. J. (2001) Chloride channel function is linked to epithelium-dependent airway relaxation. Am. J. Physiol. Lung Cell Mol Physiol. 280; L334-L341

Gallwitz, B., Ropeter, T., Morys-Wortmann, C., Mentlein, R., Siegel, E. G., Schmidt, W. E. (2000) GLP-1-analogues resistant to degradation by dipeptidyl-peptidase IV in vitro. Regul Pept 86; 103-11

Gardner, J. D., Conlon, T. P., Fink, M. L., Bodanszky, M. (1976) Interaction of peptides related to secretin with hormone receptors on pancreatic acinar cells. Gastroenterology 71:965-70

Gardner, J. D., Rottman, A. J., Natarajan, S. & Bodansky, M. (1979) Interaction of secretin 5-27 and its analogues with hormone receptors on pancreatic acini. Biochim Biophys Acta. 583; 491-503.

Gespach, C., Bataille, D., Vauclin, N., Moroder, l., Wonsch, E., Rosselin, G. (1986) Secretin receptor activity in rat gastric glands. Binding studies, cAMP generation and pharmacology. Peptides 7; 155-163

Gourlet, P., Rathé, J., De Neef, P., Chudde, J., Vandermeers-Piret, M. C., Waelbroeck, M., Robberecht, P. (1998) Interaction of lipophilic VIP derivatives with recombinant VIP1/PACAP and VIP2/PACAP receptors. Eur J Pharmacol. 354; 105-11

Haffer, B. M., Hocart, S. J., Coy, D. H., Mantey, S., Chiang, H. C. & Jensen, R. T. (1991) Reduced peptide bond pseudopeptide analogues of secretin. A new class of secretin receptor antagonists. J. Biol. Chem. 266; 316-322.

Jiang J G, Johnson C, Zarnegar R. (2001) PPAR gamma-mediated transcriptional upregulation of the hepatocyte growth factor gene promoter via a novel composite cis-acting element. J Biol Chem. Apr 5.

Konig, W., Bickel, M., Karch, K., Teetz, V. & Uhmann. (1984) Analogues and fragments of secretin. Peptides 5; 189-193.

Konig, W., Bickel, Wissmann, H., Sandeur, J. (1986) New analogues of secretin. Peptides 7; 61-67

Konig et al (Gastroenterology, 1977, 72;797-800)

Lewis B S, Flugelman M Y, Weisz A, Keren-Tal I, Schaper W. (1997) Angiogenesis by gene therapy: a new horizon for myocardial revascularization? Cardiovasc Res. 35: 490-7.

Morse M A.(2001) Technology evaluation: VEGF165 gene therapy, Valentis Inc. Curr Opin Mol Ther. 3; 97-101. Review.

Mutoh, H., Fung, B. P., Naya, F. J., Tsai, M. J., Nishitani, J. & Leiter, A. B (1997) The basic helix-loop-helix transcription factor BETA/NeuroD is expressed in mammalian enterendocrine cells and activates secretin gene expression. Proc. Natl. Acad. Sci. 94; 3560-3564.

Mutt, V., Jorpes, J. E. Magnusson, S. (1970) Structure of porcine secretin. The amino acid sequence. Eur. J. Biochem; 15; 513-519

Ng, S. S., Pong, R. T., Chow, B. K., Cheng, C. H. (1999.) Real time evaluation of human secretin receptor activity using cytosensor microphysiometry. J. Cell. Biochem. 72; 517-527

Robberecht, P., De Neef, P., Waelbroeck, M., Conius, J. C., Scemama, J. L., Fourmy, D., Pradayrol, L., Vaysse, N., Christophe, J. (1988) Secretin receptors in human pancreatic membrnaes. Pancreas 3; 529-535

Schmidt W E, Seebeck J, Höcker M, Schwarzhoff R, Schäfer H, Fornefeld H, Morys-Wortmann C, Fölsch U R, Creutzfeldt W. (1993) PACAP and VIP stimulate enzyme secretion in rat pancreatic acini via interaction with VIP/PACAP-2 receptors: additive augmentation of CCK/carbachol-induced enzyme release. Pancreas 8; 476-87

Sexton, P. M. (1999) Recent advances in our understanding of peptide hormone receptors and RAMPS. Curr. Opin. Drug Disc. Dev. 2; 440-448

Staun-Olsen, P., Ottesen, B., Gammeltoft, S. & Fahrenkrug, J. (1986) VIP binding sites on synaptosomes from rat cerebral cortex: structure-binding relationship. Peptides 7 Suppl 1; 181-186.

Suzuki S, Kawai K, Ohashi S, Mukai H, Yamashita K (1989) Comparison of the effects of various C-terminal and N-terminal fragment peptides of glucagon-like peptide-1 on insulin and glucagon release from the isolated perfused rat pancreas. Endocrinology 125;3109-14

Turner, P. R., Bambino, T. & Nissenson, R. A. (1996) A putative selectivity filter in the G-protein-coupled receptors for parathyroid hormone and secretin. J.Biol. Chem. 271; 9205-9208

Waelbroeck, M., Robberecht, P., De Neef, P., Chatelain, P. & Christophe, J. (1981) Binding of vasoactive intestinal peptide and its stimulation of adenylate cyclase through two classes of receptors in rat liver membranes. Effect of 12 secretin analogues and 12 secretin fragments. Biochim Biophys Acta 678; 83-90.

West J, Rodman D M. (2001) Gene therapy for pulmonary diseases. Chest. 119; 613-7.

Xue, W.,.Chey, W. Y., Sun, Q., Chang, T. M. (1993) Characterisation of secretin release in secretin cell-enriched preparation isolated from canine duodenal mucosa. Dig Dis Sci. 38; 344-52

Yanaihara, N., Kubota, M., Sakagami, M., Sato, H., Mochizuki, T. (1977) Synthesis of phenolic group containing analogues of porcine secretin and their immunological properties. J Med Chem 20; 648-55

Yang Y, Quitschke W W, Brewer G J. (1998) Upregulation of amyloid precursor protein gene promoter in rat primary hippocampal neurons by photbol ester, IL-1 and retinoic acid, but not by reactive oxygen species. Brain Res Mol Brain Res. 60; 40-9.

1. A method of treatment of asthma in a patient suffering from asthma, the method comprising administering to said patient an effective amount of a mammalian secretin or analogue thereof having from 1 to 7 substitutions or deletions having and retaining the ability to trigger anion efflux in respiratory tissue via the activation of a secretin receptor.

2. The method of claim 1 wherein said agent is a mammalian secretin.

3. The method of claim 1 wherein said agent is human secretin.

4. The method of claim 1 wherein said agent is administered by inhalation.

5. A composition comprising (i) a mammalian secretin or analogue thereof having from 1 to 7 substitutions or deletions having and retaining the ability to trigger anion efflux in respiratory tissue via the activation of a secretin receptor,
together with (ii) at least one other compound active against asthma.

6-10. (canceled).

11. The method of claim 1 wherein said agent comprises the sequence of SEQ ID NO:7.

12. The method of claim 3 wherein said agent is administered by inhalation.

13. The composition of claim 5 wherein the agent comprises the sequence of SEQ ID NO:7.


SEQUENCE LISTING (9 sequences)

SEQ ID NO: 1 - DNA, 22 bp, Artificial Sequence
  Description of Artificial Sequence Primer
gaccagcatcatctgagaggct

SEQ ID NO: 2 - DNA, 20 bp, Artificial Sequence
  Description of Artificial Sequence Primer
ccttcgcaggacctctcttg

SEQ ID NO: 3 - DNA, 24 bp, Artificial Sequence
  Description of Artificial Sequence Probe
tctctgtccgtgggtgaccctgct

SEQ ID NO: 4 - DNA, 22 bp, Artificial Sequence
  Description of Artificial Sequence Primer
gaaggtgaaggtcggagtcaac

SEQ ID NO: 5 - DNA, 22 bp, Artificial Sequence
  Description of Artificial Sequence Primer
cagagttaaaagcagccctggt

SEQ ID NO: 6 - DNA, 20 bp, Artificial Sequence
  Description of Artificial Sequence Probe
tttggtcgtattgggcgcct

SEQ ID NO: 7 - protein, 27 aa, Homo sapiens
HSDGTFTSELSRLREGARLQRLLQGLV

SEQ ID NO: 8 - protein, 27 aa, Sus sp.
HSDGTFTSELSRLRDSARLQRLLQGLV

SEQ ID NO: 9 - protein, 27 aa, Canis sp.
HSDGTFTSELSRLRESARLQRLLQGLV